(19) United States
(12) Patent Application Publication
(10) Pub. No.: US 2025/0288527 A1
(43) Pub. Date: Sep. 18, 2025

(54) DEVICES AND METHODS TO IMPROVE BIOAVAILABILITY OF THERAPEUTIC AGENTS

(71) Applicant: Nano Precision Medical, Inc., Alameda, CA (US)

(72) Inventors: Wouter Roorda, Alameda, CA (US); Adam Mendelsohn, Alameda, CA (US)

(73) Assignee: Nano Precision Medical, Inc., Alameda, CA (US)

(21) Appl. No.: 18/861,360

(22) PCT Filed: May 30, 2023
(86) PCT No.: PCT/US2023/023823

§ 371 Date: Oct. 29, 2024

Related U.S. Application Data
(60) Provisional application No. 63/348,754, filed on Jun. 3, 2022.

(51) Int. Cl. A61K 9/48; A61K 9/00; A61K 31/573; A61K 38/16

(52) U.S. Cl. CPC A61K 9/4808; A61K 9/0024; A61K 9/4816; A61K 9/4866; A61K 31/573; A61K 38/16

(57) ABSTRACT

The disclosure provides a device for sustained release of a therapeutic agent, comprising a capsule configured for implantation and having a reservoir; a nanoporous membrane with a plurality of pores; the therapeutic agent disposed within the reservoir; wherein the nanoporous membrane provides a diffusion path for the therapeutic agent out of the reservoir; and the device further comprising an anti-inflammatory agent.

### CROSS-REFERENCES TO RELATED APPLICATIONS

This application claims priority to U.S. Provisional Patent Application No. 63/348,754, filed Jun. 3, 2022, the contents of which are hereby incorporated by reference in their entirety for all purposes.

BACKGROUND

Many subjects, humans as well as animals, are in need of long-term treatment with therapeutic agents. In order to improve adherence, many subjects would benefit from the adherence provided by an implantable device releasing a desired therapeutic agent at a desired rate for an extended period of time.

Despite many years of research there is still a need for the development of such devices, and specifically for methods to control the rate of release of therapeutic agents from such devices upon implantation in a subject to be treated. The present disclosure satisfies this need and offers other advantages as well.

### BRIEF SUMMARY

In one embodiment, the present disclosure provides a device for sustained release of a therapeutic agent, the device comprising:
a capsule configured for implantation and having a reservoir;
a nanoporous membrane with a plurality of pores;
the therapeutic agent disposed within the reservoir; and
wherein the nanoporous membrane provides a diffusion path for the therapeutic agent out of the reservoir; and the device further comprising an anti-inflammatory agent.

The device as above, wherein the anti-inflammatory agent is contained within the reservoir of the device.

The device as above, wherein the anti-inflammatory agent is disposed on the outer surface of the capsule.

The device as above, wherein the anti-inflammatory agent is a steroid and/or an NSAID.

The device as above, wherein the anti-inflammatory agent is a biocompatible polymer.

The device as above, wherein the anti-inflammatory agent is in at least two different locations relative to the capsule.

In certain aspects, a polymeric stabilizing agent is disposed within the reservoir and comprises an insoluble polymer having a plurality of pH sensitive stabilizing groups. Optionally, the polymeric stabilizing agent has dimensions larger than the pore size of the nanoporous membrane substantially preventing release of the polymeric stabilizing agent from the reservoir.

In another embodiment, the disclosure provides a method for improving plasma levels of a therapeutic agent released from a device for sustained release of the therapeutic agent, comprising:
providing the device, the device comprising:
a capsule configured for implantation and having a reservoir;
a nanoporous membrane with a plurality of pores;
the therapeutic agent disposed within the reservoir;
wherein the nanoporous membrane provides a diffusion path for the therapeutic agent out of the reservoir; and the device further comprising an anti-inflammatory agent,
wherein the anti-inflammatory agent improves the plasma levels of the therapeutic agent upon implantation of the device in a subject.

The method as above, wherein the anti-inflammatory agent is contained within the reservoir of the device.

The method as above, wherein the anti-inflammatory agent is disposed on the outer surface of the capsule.

The method as above wherein the anti-inflammatory agent is a steroid and/or an NSAID.

The method as above, wherein the anti-inflammatory agent is a biocompatible polymer.

In certain aspects, a polymeric stabilizing agent is disposed within the reservoir and comprises an insoluble polymer having a plurality of pH sensitive stabilizing groups. Optionally, the polymeric stabilizing agent has dimensions larger than the pore size of the nanoporous membrane substantially preventing release of the polymeric stabilizing agent from the reservoir.

In still yet another embodiment, the present disclosure provides a method for treating a subject with a therapeutic agent and an anti-inflammatory agent, comprising:
providing a device, the device comprising:
capsule configured for implantation and having a reservoir;
a nanoporous membrane with a plurality of pores;
the therapeutic agent disposed within the reservoir; wherein the nanoporous membrane provides a diffusion path for the therapeutic agent out of the reservoir;
the device further comprising an anti-inflammatory agent; and
implanting the device in the subject.

The method as above, wherein the anti-inflammatory agent is contained within the reservoir of the device.

The method as above, wherein the anti-inflammatory agent is disposed on the outer surface of the capsule.

The method as above, wherein the anti-inflammatory agent is a steroid and/or an NSAID.

The method as above, wherein the anti-inflammatory agent is a biocompatible polymer.

In certain aspects, a polymeric stabilizing agent is disposed within the reservoir and comprises an insoluble polymer having a plurality of pH sensitive stabilizing groups. Optionally, the polymeric stabilizing agent has dimensions larger than the pore size of the nanoporous membrane substantially preventing release of the polymeric stabilizing agent from the reservoir.

In another embodiment, the disclosure provides a method for improving plasma levels of a therapeutic agent released from a device for sustained release of the therapeutic agent, comprising:
providing the device, the device comprising:
a capsule configured for implantation and having a reservoir;
a nanoporous membrane with a plurality of pores;
the therapeutic agent disposed within the reservoir; wherein the nanoporous membrane provides a diffusion path for the therapeutic agent out of the reservoir; and
co-administering an anti-inflammatory agent.

The method as above, wherein the anti-inflammatory agent is co-administered locally.

The method as above, wherein the anti-inflammatory agent is co-administered systemically.

The method as above, wherein the anti-inflammatory agent is a steroid and/or an NSAID.

In certain aspects, a polymeric stabilizing agent is disposed within the reservoir and comprises an insoluble polymer having a plurality of pH sensitive stabilizing groups. Optionally, the polymeric stabilizing agent has dimensions larger than the pore size of the nanoporous membrane substantially preventing release of the polymeric stabilizing agent from the reservoir.

In still another embodiment, the present disclosure provides a method for treating a subject with a therapeutic agent and an anti-inflammatory agent, comprising:
providing a device, the device comprising:
a capsule configured for implantation and having a reservoir;
a nanoporous membrane with a plurality of pores;
the therapeutic agent disposed within the reservoir;
wherein the nanoporous membrane provides a diffusion path for the therapeutic agent out of the reservoir;
implanting the device in the subject, and
co-administering an anti-inflammatory agent.

The method as above, wherein the anti-inflammatory agent is co-administered locally.

The method as above, wherein the anti-inflammatory agent is co-administered systemically.

The method of as above, wherein the anti-inflammatory agent is a steroid and/or an NSAID.

In certain aspects, a polymeric stabilizing agent is disposed within the reservoir and comprises an insoluble polymer having a plurality of pH sensitive stabilizing groups. Optionally, the polymeric stabilizing agent has dimensions larger than the pore size of the nanoporous membrane substantially preventing release of the polymeric stabilizing agent from the reservoir.

In yet another embodiment, the present disclosure provides a method for improving plasma levels of a therapeutic agent released from a device for sustained release of the therapeutic agent, comprising:
providing the device, the device comprising:
a capsule configured for implantation and having a reservoir;
a nanoporous membrane with a plurality of pores;
the therapeutic agent disposed within the reservoir;
wherein the nanoporous membrane provides a diffusion path for the therapeutic agent out of the reservoir;
the device further comprising an anti-inflammatory agent, and wherein the anti-inflammatory agent improves the AUC of the therapeutic agent upon implantation of the device in a subject.

In certain aspects, the AUC is increased by at least 10%, or by at least 25%, or by at least 50%, or by at least 75%, or the AUC is at least doubled.

The method of as above, wherein the anti-inflammatory agent is a steroid and/or an NSAID.

In certain aspects, a polymeric stabilizing agent is disposed within the reservoir and comprises an insoluble polymer having a plurality of pH sensitive stabilizing groups. Optionally, the polymeric stabilizing agent has dimensions larger than the pore size of the nanoporous membrane substantially preventing release of the polymeric stabilizing agent from the reservoir.

These and other aspects, objects and embodiments will be more apparent when read with the detailed description and drawings which follow.

### BRIEF DESCRIPTION OF THE DRAWINGS

FIG. 1 illustrates a device of the disclosure.

FIG. 2 illustrates an embodiment of the present disclosure.

FIG. 3 illustrates an embodiment of the present disclosure showing dexamethasone release.

FIG. 4 illustrates an embodiment of the present disclosure, showing the effect of dexamethasone in a rat model.

FIG. 5 illustrates an alternative embodiment of the device, showing the effect of dexamethasone in a cat model.

### DETAILED DESCRIPTION

The disclosure pertains to the field of long-term treatment of subjects with implantable devices providing a sustained delivery of therapeutic agents at a controlled rate.

Embodiments of the disclosure include devices, formulations, and methods to control the rate of release of therapeutic agents from such devices.

Additionally, embodiments of the disclosure include methods of treatment of a subject with devices and formulations of the disclosure.

Implantable devices with nanoporous membranes for the release of therapeutic agents have been described previously, e.g. in U.S. Pat. Nos. 9,814,867 and 9,770,412 and US Patent Publication Nos. US2022/0008345 and US2021/0246271 and US 2017/0136224, each of the foregoing incorporated herein by reference.

### Definitions

“Polypeptides” refers to molecules with a backbone chain of 2 or more amino acid residues. Some polypeptides may have additional associated groups, such as metal ions in metalloproteins, small organic molecules such as in heme proteins, or carbohydrate groups such as in glycoproteins.

“Peptides” and “Proteins” refers to subgroups of polypeptides. In this disclosure the definition of peptides and proteins follows the practice of the United States Food and Drug Administration, the FDA, which defines peptides as polypeptides with up to 40 amino acid residues, and proteins as polypeptides with more than 40 amino acid residues.

Incretin mimetics refers to agents that act like incretin hormones such as glucagon-like peptide-1 (GLP-1). They bind to GLP-1 receptors and stimulate glucose dependent insulin release, therefore acting as antihyperglycemics.

Exenatide (natural, recombinant and synthetic, also called exendin-4) refers to amino acid sequence His Gly Glu Gly Thr Phe Thr Ser Asp Leu Ser Lys Gln Met Glu Ala Val Arg Leu Phe Ile Glu Trp Leu Lys Asn Gly Pro Ser Gly Ala Pro Ser.

“Formulation of a therapeutic agent” refers to the actual state in which a therapeutic agent is present in a product or in a product fabrication intermediate, and includes the therapeutic agent, plus, optionally, any used additional therapeutic agents, any used formulation excipients and any used formulation solvents.

“Membrane” refers to a permeable structure allowing mass transport of molecules from one side of the structure to the other through the structure.

“Porous membranes” refers to membranes characterized by the presence of a two-phase system, in which membrane matrix material represents one phase, typically a continuous phase, which is permeated by open channels extending from one side of the membrane to the other, and filled with a second phase, often a fluid phase, through which mass transport through the membrane can take place.

“Dense” or “non-porous membranes” refers to membranes without fluid filled pores. In such membranes mass transport may take place by a dissolution-diffusion mechanism, in which therapeutic agents permeate the membrane by dissolving in the membrane material itself, and diffusing through it.

“Nanoporous membrane” and “nanopore membrane” are used interchangeably, and refer to porous membranes in which the pores have a smallest diameter of less than 1000 nanometer.

“Nanotube membrane” refers to a nanoporous membrane, wherein pores are formed by an array of nanotubes.

“Titania nanotube membrane” refers to an array of titania nanotubes on a titanium substrate where at least a portion of the titania nanotubes are open at both ends and capable of allowing diffusion from one side of the membrane to the other through the titania nanotubes. In certain instances, the titania nanotube membrane has two faces or sides. A first face or side having an array of titania nanotubes and a second face or side of a titanium substrate. In certain aspects, the array of titania nanotubes are grown on the titanium substrate by electrochemical anodization.

“Molecular diameter” of a polymer refers to the diameter of the sphere of gyration of the polymer, which is a physical measure of the size of a molecule, and is defined as two times the mass weighted average distance from the core of a molecule to each mass element in the molecule.

“Stokes diameter” or “hydrodynamic diameter” refers to the dimension of a molecule plus its associated water molecules as it moves through an aqueous solution, and is defined as the radius of an equivalent hard sphere diffusing at the same rate as the molecule under observation.

“Ion exchange resin” refers to an insoluble polymer comprising acidic or basic groups, or a combination thereof, made insoluble, for instance by cross-linking, and capable of exchanging anions or cations, or a combination thereof, with a medium surrounding it.

“Fluid” and “fluid form” as used in this disclosure refers to flowable states of matter and includes, but is not limited to, gases, solutions, suspensions, emulsions, colloids, dispersions and the like.

“Fluid contact” refers to an entity being in contact with a fluid.

“Neutral pH” refers to a pH between 6 and 8 such as between 6.5 and 7.5.

Implantable devices with nanoporous membranes for the release of therapeutic agents have been described previously, e.g. in U.S. Pat. Nos. 9,814,867, 9,770,412 and 11,129,791. and WO 2021/173770, each of the foregoing incorporated herein by reference. It has now been found that the release rate of the therapeutic agents from these devices can be powerfully controlled by the pH of the formulations of the therapeutic agent. Some embodiments of the disclosure include a device with a cylindrical capsule encapsulating a reservoir, a nanoporous membrane affixed to one end of the capsule, and a formulation of a therapeutic agent contained within the reservoir. Release of the therapeutic agent from the reservoir after implantation of the device in a subject is controlled by the nanoporous membrane. Some embodiments of the disclosure utilize the pH of the formulation of the therapeutic agent as a further means to control the release rate. Additionally, some embodiments control the duration of release of the therapeutic agent using the orientation of the membrane with respect to the reservoir.

### Devices

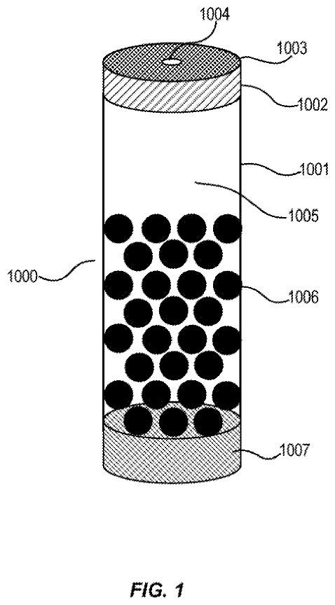

As illustrated in FIG. 1, devices of the disclosure include a capsule 1000 suitable for implantation, wherein the capsule has a reservoir 1001 suitable for holding a therapeutic agent 1005 and, optionally, a pH controlling agent in the form of resin beads 1006 (e.g., an insoluble polymer having a plurality of pH sensitive stabilizing groups). In some embodiments more than one reservoir is present.

The capsule 1000 may be made of any suitable biocompatible material. In some embodiments the capsule is made of a medical grade metal, such as titanium or stainless steel, or of a medical grade polymeric material, such as silicone, polyurethane, polyacrylate, polyolefin, polyester, polyamide and the like. In some embodiments the capsule is made of multiple materials. In some embodiments of the disclosure, the capsule is made of titanium.

Devices of the disclosure have at least one membrane 1004, as described herein, attached to the capsule and in fluid contact with the reservoir, wherein the membrane provides a pathway for mass transport of a therapeutic agent included within the reservoir 1001 out of that reservoir and into the body of a subject into which the capsule has been implanted. In this disclosure “attached to the capsule” refers to a component being fixed in place with respect to the capsule, and connected to the capsule directly or indirectly, by using any suitable means, including by welding, gluing, press-fitting and by using threaded means, or by any combination of these.

In the case of membranes as described in U.S. Pat. No. 9,814,867, and as illustrated in FIG. 1, the nanotube membranes are part of an array of nanotubes 1003, some of which are still attached to the titanium substrate 1002 from which they were grown, and the substrate may be attached to the capsule. At least some of the nanotubes are open on both sides, to allow for mass transport of a therapeutic agent out of the reservoir. In one aspect, the membrane 1004 is a titania nanotube membrane 1004, which has two faces or sides. A first face or side having an array of titania nanotubes 1003 and a second face or side of a titanium substrate 1002. FIG. 1 shows the membrane attached to the capsule with the titanium substrate 1002 facing towards the reservoir of the device. Some devices of the disclosure further have a septum 1007, pierceable with a needle, and suitable as access port to deposit formulation 1005 into the reservoir 1001.

Further description of devices of the disclosure may be found in WO 2021/173770 incorporated herein by reference.

### Membranes

Embodiments of the disclosure include at least one membrane providing a pathway for mass transport of a therapeutic agent out of a reservoir of a device of the disclosure.

A wide variety of membranes can be used in embodiments of the present disclosure.

Membranes of the disclosure include dense and porous membranes; porous membranes include nanoporous membranes and nanotube membranes.

Suitable materials for membranes of the disclosure include organic and inorganic materials, polymers, ceramics, metals, metal oxides and combinations thereof. Suitable materials for the membrane include silicon, silica, titanium and titania.

In some embodiments, the membrane is a nanoporous membrane. In some embodiments the membrane is a nanotube membrane. In some embodiments the membrane is a titania nanotube membrane.

Embodiments of the disclosure are particularly useful as sustained delivery devices for therapeutic agents, in which the release of the agents is controlled by a nanoporous membrane.

Fabrication of membranes of the disclosure is described in U.S. Pat. No. 9,814,867 and control of the internal diameter of the nanopores is described in U.S. Pat. No. 9,770,412, each incorporated herein by reference.

Further description of membranes of the disclosure may be found in WO 2021/173770 incorporated herein by reference.

### Formulations

Devices of the disclosure include a formulation having at least one therapeutic agent, for instance therapeutic agents such as described in this disclosure. The therapeutic agent may be in solid or fluid form. In some instances, the therapeutic agent may be present in mixed forms, such a suspension of a solid form of the therapeutic agent in a saturated solution of the therapeutic agent. In some instances, the formulation is in solid form, in some instances the formulation is in fluid form. Formulations in fluid form, for instance formulations including a solution of at least part of the therapeutic agent in the reservoir, may have a pH.

### pH Controlling Agents

Materials to control the pH may be the therapeutic agent itself, low molecular weight stabilizers, such as acidic and basic compounds, including weakly acidic and weakly basic compounds that can be used as buffering agent, or high molecular weight compounds like poly-acids or poly-bases. Many such compounds are known in the literature, and those with ordinary skills in the art of pharmaceutical formulation development will be able to select suitable ingredients for the formulation without undue experimentation.

In some embodiments the pH controlling materials are insoluble polymeric stabilizers as described in WO 2021/173770 incorporated herein by reference. Other pH controlling agents suitable for the disclosure can be found in U.S. Pat. Nos. 10,045,943, and 10,479,868, incorporated herein by reference. Ion exchange resin is an example of an insoluble polymer having a plurality of pH sensitive stabilizing groups.

“Acid” refers to a compound that is capable of donating a proton (H+) under the Bronsted-Lowry definition, or is an electron pair acceptor under the Lewis definition. Acids useful in the present disclosure are Bronsted-Lowry acids that include, but are not limited to, alkanoic acids or carboxylic acids (formic acid, acetic acid, citric acid, lactic acid, oxalic acid, etc.), sulfonic acids and mineral acids, as defined herein. Mineral acids are inorganic acids such as hydrogen halides (hydrofluoric acid, hydrochloric acid, hydrobromic acid, etc.), halogen oxoacids (hypochlorous acid, perchloric acid, etc.), as well as sulfuric acid, nitric acid, phosphoric acid, chromic acid and boric acid. Sulfonic acids include methanesulfonic acid, benzenesulfonic acid, p-toluenesulfonic acid, triflouromethanesulfonic acid, among others.

“Base” refers to a compound capable of accepting a proton (H+) under the Bronsted-Lowry definition, or is an electron pair donor under the Lewis definition. Representative bases include, but are not limited to, hydroxy, alkylhydroxy, amines (—NRR), alkylamine, arylamine, amide (—C(O)NRR), sulfonamide (—S(O)₂NRR), phosphonamide (—P(O)(—NRR)₂), carboxylate (—C(O)O—), and others.

In certain instances, the pH adjusting agent is a buffer. The buffer is selected from the group consisting of citrate/citric acid, acetate/acetic acid, phosphate/phosphoric acid, formate/formic acid, propionate/propionic acid, lactate/lactic acid, carbonate/carbonic acid, ammonium/ammonia, edentate/edetic acid, and combinations thereof.

Therapeutic agents

Therapeutic agents suitable for embodiments of the disclosure have been described in WO 2021/173770 incorporated herein by reference. In some embodiments the therapeutic substance is a peptide or protein. In some embodiments the peptide or protein is an incretin mimetic. In some embodiments the incretin mimetic is exenatide.

### Manufacture

Methods of manufacture of devices and formulations are described in WO 2021/173770 incorporated herein by reference.

### Release Rate Control

Devices of the disclosure have the capability to release therapeutic agents, contained in the reservoir, through the nanopores of the membrane at a controlled rate. In some instances, the rate of release of the therapeutic agent is a non-Fickian release rate, i.e., a release rate that is not proportional to the concentration gradient driving the release (e.g., zero-order release). Examples of non-Fickian release rates through nanoporous membranes have been described in U.S. Pat. No. 9,814,867, incorporated herein by reference.

The exact mechanism by which the nanopores of the membranes control the release rate is not understood in detail. Hypothetically, interactions between the diffusing molecules of the therapeutic agent and the interior wall of the nanotubes could play a role in this mechanism.

WO 2021/173770 incorporated herein by reference, discloses the use of insoluble polymeric agents with a plurality of pH sensitive stabilizing groups that can be employed to provide buffering capacity at desirable pH levels, such as weakly acidic or weakly basic groups, to provide chemical stabilization for therapeutic agents in devices of the disclosure. These polymeric agents stabilize the therapeutic agents by controlling the pH of formulations of the disclosure. Serendipitously, such chemical stabilizers can now be used to control release rates as well. Ion exchange resin is an example of an insoluble polymer having a plurality of pH sensitive stabilizing groups.

Crosslinked poly-acrylic acid and poly-methacrylic acid are used commercially as ion exchange resins. In some embodiments of the disclosure, ion exchange resins are used as stabilizing agents. Potentially suitable ion exchange resins are produced by Mitsubishi Chemical Corporation under the name “Diaion” and by Purolite Corporation under the name “Purolite”. In some instances, Diaion WK40L and Purolite 104plus and Purolite C115 may be suitable stabilizing agents for the stabilization of peptide and protein formulations. Others include Diaion WK10, Diaion WK11, Diaion WK100, and Diaion WT01S.

Therefore, embodiments of the disclosure offer a method of controlling the release rate of a therapeutic agent through a nanoporous membrane by adjusting the pH of the formulation of the therapeutic agent.

Some embodiments of the disclosure provide methods to control the rate of release of therapeutic agents from a reservoir through a nanotube membrane by controlling the pH of a formulation in the reservoir in which at least part of the therapeutic agent, or therapeutic agents, is dissolved. In some embodiments the release rate is controlled by controlling the pH with polymeric stabilizers such as described in WO 2021/173770 incorporated herein by reference.

In some embodiments, the release rate is controlled by controlling the pH with soluble pH controlling stabilizers, such as low molecular weight acids or bases. In some instances, a gradual rise of the release rate of a drug from an implant over time is considered desirable. For instance, with exenatide a gradual ramp-up of the delivered dose per day has been associated with a reduced incidence of nausea. In some embodiments of the disclosure the initial internal pH of a device is set at a relatively low level, and is allowed to rise over time as the internal pH slowly equilibrates with the external environment of the device, i.e. interstitial fluid. The gradual rise in pH is accompanied by a gradual increase in release rate.

In some instances, a dry formulation of a therapeutic agent may be present in a device at the time of implantation in a subject. In such instances a promotor of water uptake may be present in the reservoir, such as a water-soluble gas. After implantation the water-soluble gas may promote the uptake of interstitial fluid into the reservoir through the membrane of the device. Embodiments of the disclosure may include a dry formulation in the reservoir with a composition that, after uptake of the interstitial fluid, generates a liquid formulation with a pH that provides a desired release rate of the therapeutic agent.

### Anti-Inflammatory Agents

In vitro, the rate of release of a therapeutic agent, e.g., exenatide, from devices of the disclosure was consistently sustained at elevated levels for extended periods time.

However, upon implantation in laboratory animals, certain titanium-based devices of the disclosure showed a significant decline in plasma levels in vivo of the therapeutic agent over similar time periods. This effect was observed in both a rat model and a cat model. In the rat model, a pH stabilizing resin (Purolite PPC115PHDR, adjusted to H 5.5 with NaOH) was included in the devices, however, in the cat model it was not included.

Soon after implantation of a device into a subject, proteins and other biomolecules present in the blood, plasma and other biological fluids rapidly adsorb onto the surface of biomaterials of the implant.

Excessive inflammation, with its expression of proteolytic and metabolic enzymes, as well as the formation of excessive tissue capsules or other structural changes are known consequences of implantation of non-biocompatible materials and may affect plasma levels of therapeutic agents negatively. However, titanium is considered a gold standard for implantable devices, given its excellent biocompatibility.

Histology on tissue samples surrounding the implanted devices in rats did not show unusual inflammation or abnormal structural changes and demonstrated the expected high degree of biocompatibility. In several cat subjects, a mild to moderate inflammatory response was observed. Therefore, while in some subjects an anti-inflammatory action of an added biologically active substance may be beneficial, other, so far unidentified factors may play a role as well.

Upon explantation of the devices at the end of the in-life implantation period, the devices were opened and the remaining formulations analyzed. In rats, exenatide is eliminated by renal clearance and little or no enzymatic metabolic degradation is observed. Accordingly, degradation products found in the devices from the rats showed the normal chemical degradation products and no sign of enzymatic degradation was found.

By contrast, in cats a specific fragment of exenatide was observed, indicating enzymatic cleavage of the peptide backbone. The presence of this fragment correlated with the decline in plasma levels of intact exenatide.

Therefore, the mechanism by which the plasma levels were suppressed appears to be different in rats than in cats, and while in cats only a partial correlation with inflammation was observed, no such correlation weas observed in rats.

Despite the absence of signs of excessive inflammation or abnormal tissue remodeling, surprisingly, the addition of an anti-inflammatory steroid, e.g., dexamethasone, did significantly increase the plasma levels of therapeutic agent released from the titanium devices in vivo. Therefore, it is believed that the significant increase in plasma levels of a therapeutic agent effectuated by anti-inflammatory drugs represents a novel mechanism to improve plasma levels of the therapeutic agent released by implantable devices.

The concept of Area Under the Curve (AUC) is fundamental to pharmacokinetic modeling of exposure of the body of a subject to administered therapeutic agents. Briefly, samples are taken from a body fluid of the subject at certain time intervals, and the concentrations of the therapeutic agent are measured. A curve is constructed, representing the concentrations in the body fluid over time. The area under the curve is a measure of the exposure of the body to the therapeutic agent.

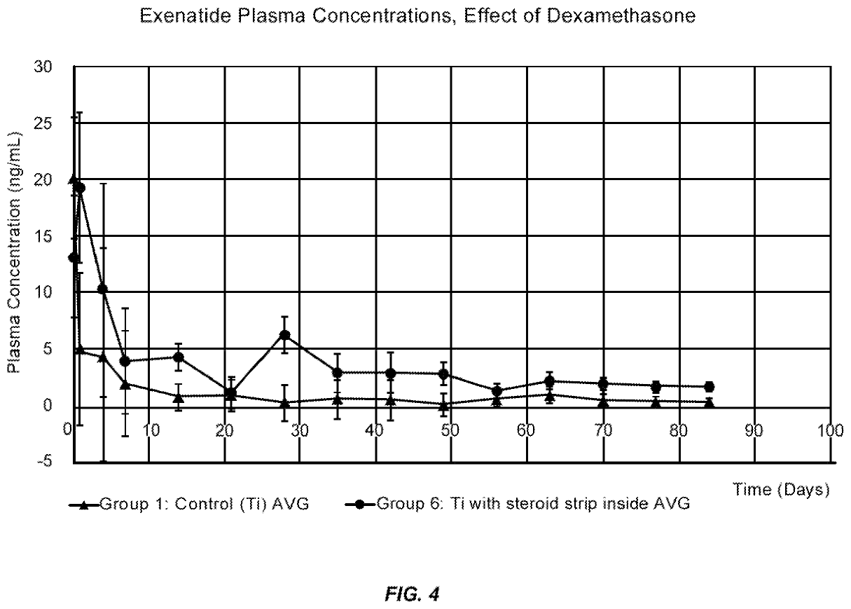

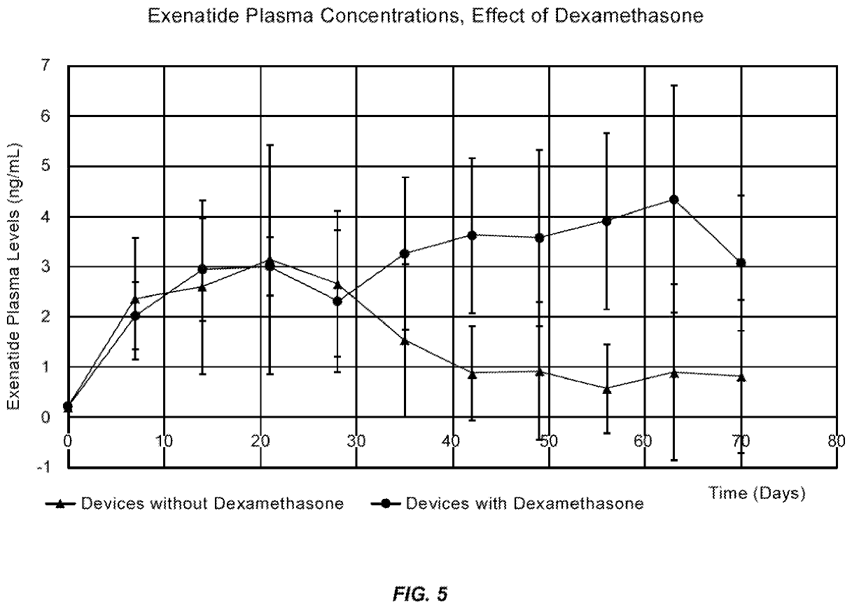

As can be seen in FIG. 4 and FIG. 5 and as is described in the examples below, the AUC resulting from an implant releasing a therapeutic agent together with an anti-inflammatory agent was significantly larger than the AUC resulting from an implant without an anti-inflammatory agent.

Embodiments of the disclosure include devices, compositions and methods to increase the AUC of a therapeutic agent in a subject upon implantation of an implantable device of the disclosure. In some embodiments, the AUC is increased by about 10% to about 100%.

In some embodiments the AUC is increased by at least 10% compared to plasma levels without the anti-inflammatory agent.

In some embodiments the AUC is increased by at least 25% compared to plasma levels without the anti-inflammatory agent.

In some embodiments the AUC is increased by at least 50% compared to plasma levels without the anti-inflammatory agent.

In some embodiments the AUC is increased by at least 75% compared to plasma levels without the anti-inflammatory agent.

In some embodiments the AUC is at least doubled compared to plasma levels without the anti-inflammatory agent.

Dexamethasone has been used to protect glucose sensors from reactive oxygen species (U.S. Pat. No. 9,931,068), to protect cardiac pacemakers from tissue overgrowth (U.S. Pat. No. 7,164,948) and as a primary treatment for ophthalmological diseases (U.S. Pat. No. 9,012,437), but has not been used before to improve plasma levels and AUCs achieved with implantable devices delivering a different therapeutic agent as the primary treatment modality. Since inflammation does not appear to play a role in the occurrence of low plasma levels of therapeutic agents delivered from devices of the embodiment, the mechanism of action of the anti-inflammatory agent is not known.

Embodiments of the disclosure may include any suitable type of anti-inflammatory agents. Anti-inflammatory agents of the disclosure include bound agents, including certain anti-inflammatory coatings, such as suitable hydrogel coatings, and released agents, such as anti-inflammatory drugs. Anti-inflammatory drugs may include any type of anti-inflammatory drug, such as steroidal anti-inflammatory drugs (“steroids”) and Non-Steroidal Anti-Inflammatory Drugs” (“NSAIDS”) and combinations thereof.

Dexamethasone is a typical and representative member of the corticosteroid class of drugs, a subclass of the steroid class. Any suitable type of steroid may be used, including, but not limited to, corticosteroids, such as cortisone, prednisone, prednisolone, methylprednisolone, dexamethasone, betamethasone, hydrocortisone, cortisone, cortisol, flunisolide, fluticasone furoate, fluticasone propionate, triamcinolone acetonide, beclomethasone dipropionate, budesonide, mometasone furoate, ciclesonide, clobetasol and clobetasol.

Any suitable type of NSAID may be used, including non-selective COX inhibitors, selective COX 1 inhibitors and selective COX 2 inhibitors.

Suitable NSAIDS include, but are not limited to, diclofenac, indomethacin, sulindac, mefenamic acid, piroxicam, ibuprofen, ketoprofen, naproxen, phenylbutazone, aspirin, diflunisal, etodolac, fenoprofen, flurbiprofen, meclofenamate, nabumetone, oxaprozin, tolmetin, meloxicam, nimesulide, celecoxib, etoricoxib, valdecoxib, celecoxib, rofecoxib, parecoxib and acetaminophen.

In certain instances, the anti-inflammatory agent is a biocompatible polymer. The biocompatible polymer may be a biodegradable or erodible polymer coating containing an anti-inflammatory drug. Other anti-inflammatory polymers include heparin, hyaluronic acid, alpha melanocyte-stimulating hormone (α-MSH), polyethylene glycol (PEG), crossed-linked PEG and polymers that inhibit pro-inflammatory cytokines.

The amount of an anti-inflammatory agent to be included in embodiments of the disclosure may vary with the intended strength and duration of the effect, and with the potency of the anti-inflammatory agent. For example, anti-inflammatory corticosteroids are available in a wide range of strengths, with hydrocortisone being a low-strengths agent, dexamethasone a moderately strong agent, and clobetasol being one of the so-called super potent agents.

Experimental models, including animal models are available to test the effect of different dose regimens of anti-inflammatory agents, and those skilled on the art of pharmaceutical development will be able to design the experiments to determine the desired type and dose regimen of the anti-inflammatory agent.

Available literature, prescribing information and public regulatory documents may be helpful in such determination. For example, and not by way of any limitation, the maximum prescription dose of oral dexamethasone is 9 mg. The recommended dose of dexamethasone as a long-acting steroid injection in small joints is 0.8-1 mg. The tip of a permanently implanted pacemaker, the Medtronic Attain Ability TM Model 4196 Lead is coated with a slow-release coating containing 160 micrograms of dexamethasone.

In some embodiments more than one anti-inflammatory agent may be present.

Embodiments of the disclosure further include implantable devices for the delivery of therapeutic agents to a subject, wherein the devices include an anti-inflammatory agent in addition to the therapeutic agent. In some instances, the anti-inflammatory agent improves the therapeutic effect of the therapeutic agent. The anti-inflammatory agent may be included in any suitable location on the device, including being present in a reservoir of the device, or being present on a coating on the outside of the reservoir.

The amount of anti-inflammatory agent is an amount sufficient to co-diffuse with the therapeutic agent. If the device has enough sustained release active agent to diffuse for 1-52 weeks, 1-12 months, so too will the anti-inflammatory agent. In certain aspects, the concentration or amount of the of the anti-inflammatory agent is dependent on the amount or concentration of the active agent.

A formulation of a therapeutic agent comprises a therapeutic agent, anti-inflammatory and optionally excipients and solvents. In certain instances, the amount of therapeutic agent is about 5% to about 40% w/w of the formulation disposed with the implantable device. The formulation may be a saline solution together with a pH modifying agent such as a buffer. The therapeutic agent can be about 15% to about 35% w/w, or about 20% to about 30%, or about 25% to about 30% w/w or about 25% w/w of the formulation.

The amount of therapeutic agent present in the device is enough for about 1 month to about 12 months for example, up to 1 month, 2, 3, 4, 5, 6, 7, 8, 9, 10, 11 or 12 months of extended release. The amount of anti-inflammatory agent in the device is an amount sufficient to co-diffuse with the therapeutic agent. If the therapeutic agent delivers extended release for 2 months, so too will the anti-inflammatory co-diffuse for 2 months. The concentration amount of anti-inflammatory agent can be more or less than the amount of therapeutic agent as long as there is an amount sufficient of the anti-inflammatory to co-diffuse for the same time frame.

In certain aspects, the therapeutic agent has a first diffusion rate through the nanoporous membrane, and the anti-inflammatory has a second diffusion rate through the nanoporous membrane. The first diffusion rate can be less than, greater than or the same as the second diffusion rate. As long as there is an amount of anti-inflammatory agent to co-diffuse with the therapeutic agent, the amount of anti-inflammatory agent is sufficient.

In certain aspects, the total potency scale is often assumed to cover a roughly 2000× range, leaving a wide range of options available in terms of quantity of the anti-inflammatory agent to be included. As a general guide, it appears that a local activity of the released anti-inflammatory agent is responsible for the beneficial effect, since very low daily doses were required. For instance, for dexamethasone, only about a quarter of a microgram (0.25 μg/day) or was measured as the daily release. In certain instances, 0.01 μg/day to about 5 μg/day can be released. Based on such considerations, and the total desired duration of the implanted device, a total loading of the therapeutic agent and anti-inflammatory agent can be determined.

Some embodiments of the disclosure include a kit, in which an implantable device for the delivery of a therapeutic agent is present, as well as a dosage form of the anti-inflammatory agent. The dosage form may be any desirable dosage form, such as encapsulated in microparticles or a solid implant, or any other form of a drug formulation with a desirable dissolution profile, such as a depot of a long-acting corticosteroid.

Embodiments of the disclosure further include methods for treating a subject with anti-inflammatory agents and for improving the plasma levels of therapeutic agents delivered from devices of the disclosure. Methods of the disclosure include administration of any of the devices or compositions of the disclosure to subject in need of such administration. Administration of the anti-inflammatory agent may be systemic or local. Systemic administration may include oral and parenteral administration, such as by injection, and trans-tissue administration, such as transdermal and transmucosal. Local administration may include inclusion of the agent in a reservoir of a device of the disclosure, either co-administered with a main therapeutic agent or from its own reservoir.

Alternatively, or concomitantly, the anti-inflammatory agent may be present on the device outside the reservoir, such as in a coating.

Further alternatively, the anti-inflammatory agent may be administered locally in its own delivery form, such as encapsulated in microparticles or in a solid implant, or in any other form of a drug formulation with a desirable dissolution profile, such as a depot of a long-acting corticosteroid. Further alternatively, the anti-inflammatory agent may be administered systemically.

Embodiments of the disclosure further include methods to improve the plasma levels of therapeutic agents released from implantable devices. Methods of the disclosure include co-formulation, co-administration, or otherwise providing for a simultaneous activity of a therapeutic agent and an anti-inflammatory agent. The action of the anti-inflammatory agent may suppress the inflammatory response to implantation of devices for the sustained release of therapeutic agents, thereby reducing degradation of the therapeutic agent by inflammatory agents, such as proteases produced by inflammatory cells.

### EXAMPLES

Dexamethasone releasing strips were manufactured by mixing 1 part dexamethasone with 2 parts of silicone precursor MED-4830 Binary Silicon until a visually uniform paste was achieved. The paste was drawn into a film of about 1 mm thick on a glass sheet, and cured for 180 minutes at 150° C. The film was then cut into strips of approximately 1×1×7 mm.

In the examples below the devices that were used included titanium capsules of approximately 25 mm length and 2.25 mm diameter. A titanium substrate with a titanium oxide nanoporous membrane was welded to one end of the device. The nanoporous membrane had a diameter of 0.3 mm and was composed of about 6,000,000 nanopores. The average diameter of the nanopores at the substrate end was approximately 20 nm. About 10 mg of ion exchange resin Purolite PPC115PHDR, adjusted to pH 5.5 with NaOH was filled into the reservoir as a pH control agent.

The groups with the anti-inflammatory agent additionally received one of the dexamethasone-loaded silicone strips as described above. A silicone septum was located at the other end of the device.

For the groups without the dexamethasone strip, about 46 mg of a formulation containing 25% exenatide-acetate (w/w), 0.25% Polysorbate 20, 154 mM Na+ and a pH of 5.5 was filled into the device as per methods in PCT/US2021/019559. For the groups with the dexamethasone strip, about 40 mg of drug formulation was loaded. Briefly, the formulation was loaded into a filler apparatus with a hollow needle to pierce the septum. A vacuum was applied to the membrane of the device to reduce the pressure inside the reservoir, and the formulation was injected through the septum into the reservoir through the needle.

### Example 1

For in vitro testing devices without a dexamethasone strip and devices with a dexamethasone strip were prepared as described above and were stored submerged in a storage buffer containing 0.9% NaCl and 0.76% sodium acetate in water for injection at pH 5.5 at room temperature before the start of release rate testing.

In vitro release rate testing was performed by submerging the devices in 3 ml of a 26 mM bis-tris buffer, 154 mM NaCl on a shaker plate at 37° C. and measuring the amounts of exenatide and dexamethasone released at regular intervals by reverse phase HPLC.

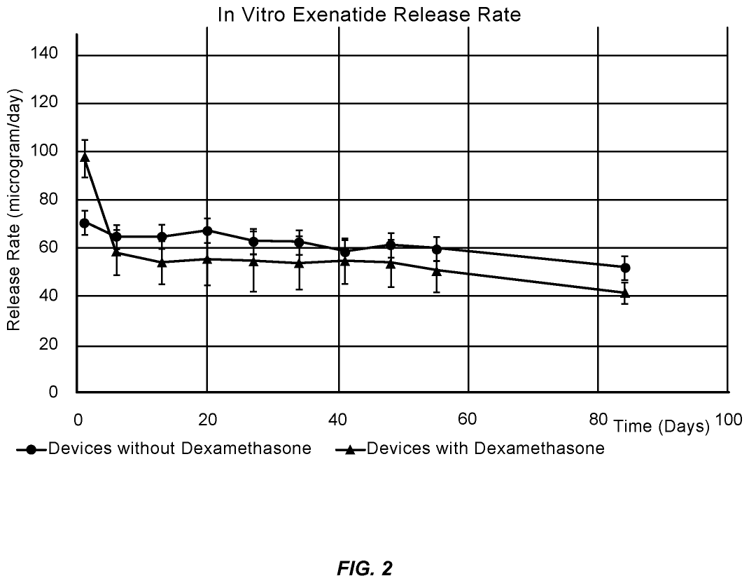

In FIG. 2, the release rates are plotted on the Y axis, and time (in days) is plotted on the X-axis. As can be seen, the release rates of the devices with the steroid strip are consistently slightly lower than those of the devices without the strip.

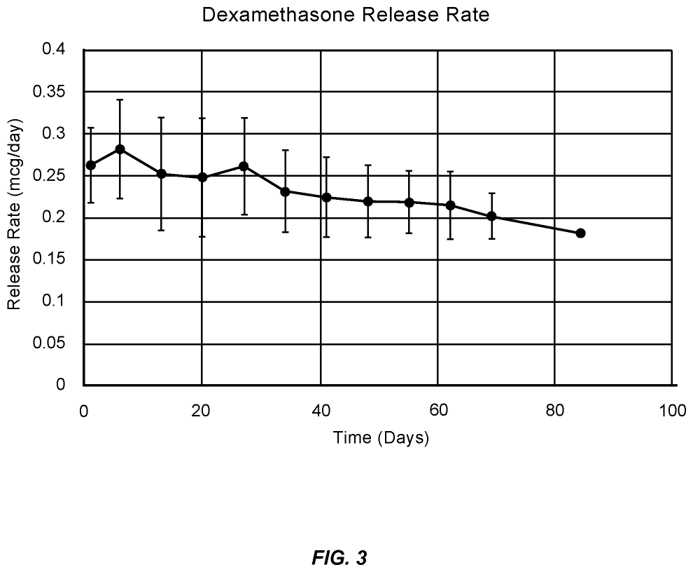

In FIG. 3, the release rates of dexamethasone from the devices are plotted in an analogous manner.

### Example 2

For in vivo testing devices without a dexamethasone strip and devices with a dexamethasone strip were prepared as described above and were stored submerged in a storage buffer containing 0.9% NaCl and 0.76% sodium acetate in water for injection at pH 5.5 at room temperature before implantation.

In one study the devices were tested by implantation in Sprague-Dawley rats and measuring the plasma concentration-time profiles. Post implantation plasma samples were collected, and exenatide plasma levels determined by LC/MS. After removal of several devices for interim analysis, and removing animals that showed signs of exenatide antibody development from the analysis, a total of 3 devices in each group was taken out to a full 84 days of implantation.

In FIG. 4, the plasma concentrations expressed in ng/ml are plotted on the Y axis, time on the X-axis. As can be seen, the devices with the dexamethasone strip showed significantly higher plasma levels than the devices without the strips.

The AUCs resulting from implantation of both devices were calculated using the trapezoid method, which is a common procedure to approximate AUCs. Briefly, each concentration point on the curve is connected by a line to its corresponding time point, thus creating a series of trapezoids. The surface areas of the individual trapezoids are calculated and added up to provide the total AUC. Following this method, the average AUC of the device without the anti-inflammatory agent was 92 ng day/mL. For the device with the anti-inflammatory agent it was 302 ng day/mL.

### Example 3

In another study the devices were tested by implantation in laboratory cats and measuring the plasma concentration-time profiles. Post implantation plasma samples were collected, and exenatide plasma levels determined by LC/MS. A total of 5 animals was included in each group.

No pH controlling resin was included in the devices implanted in these groups.

In FIG. 5, the plasma concentrations expressed in ng/ml are plotted on the Y axis, time on the X-axis. As can be seen, the devices with the dexamethasone strip showed significantly higher plasma levels than the devices without the strips.

The AUCs resulting from implantation of both devices were calculated using the trapezoid method described above. Following this method, the average AUC of the devices without the anti-inflammatory agent was 112 ng day/mL. For the devices with the anti-inflammatory agent, it was 214 ng day/mL.

The foregoing patents and patent publications are each incorporated herein by reference.

1. A device for sustained release of a therapeutic agent, the device comprising:
a capsule configured for implantation and having a reservoir;
a nanoporous membrane with a plurality of pores;
the therapeutic agent disposed within the reservoir; and
wherein the nanoporous membrane provides a diffusion path for the therapeutic agent out of the reservoir; and the device further comprising an anti-inflammatory agent.

2. The device of claim 1, wherein the anti-inflammatory agent is contained within the reservoir of the device.

3. The device of claim 1, wherein the anti-inflammatory agent is disposed on the outer surface of the capsule.

4. The device of claims 1, wherein the anti-inflammatory agent is in at least two different locations.

5. The device of claim 1, wherein the anti-inflammatory agent is a member selected from the group consisting of a steroid, an NSAID and a combination thereof.

6. The device of claim 3, wherein the anti-inflammatory agent is a biocompatible polymer.

7. A method for improving plasma levels of a therapeutic agent released from a device for sustained release of the therapeutic agent, comprising:
providing the device, the device comprising
a capsule configured for implantation and having a reservoir;
a nanoporous membrane with a plurality of pores;
the therapeutic agent disposed within the reservoir;
wherein the nanoporous membrane provides a diffusion path for the therapeutic agent out of the reservoir; and the device further comprising an anti-inflammatory agent, wherein the anti-inflammatory agent improves the plasma levels of the therapeutic agent upon implantation of the device in a subject, or co-administering an anti-inflammatory agent, or the device further comprising an anti-inflammatory agent, and wherein the anti-inflammatory agent improves the AUC of the therapeutic agent upon implantation of the device in a subject.

8. The method of claim 7, wherein the anti-inflammatory agent is contained within the reservoir of the device.

9. The method of claim 7, wherein the anti-inflammatory agent is disposed on the outer surface of the capsule.

10. The method of claim 7, wherein the anti-inflammatory is a member selected from the group consisting of a steroid, an NSAID and a combination thereof.

11. The method of claim 9, wherein the anti-inflammatory agent is a biocompatible polymer.

12. A method for treating a subject with a therapeutic agent and an anti-inflammatory agent, comprising:
providing a device, the device comprising
capsule configured for implantation and having a reservoir;
a nanoporous membrane with a plurality of pores;
the therapeutic agent disposed within the reservoir;
wherein the nanoporous membrane provides a diffusion path for the therapeutic agent out of the reservoir;
the device further comprising an anti-inflammatory agent, or co-administering an anti-inflammatory agent; and
implanting the device in the subject.

13. The method of claim 12, wherein the anti-inflammatory agent is contained within the reservoir of the device.

14. The method of claim 12, wherein the anti-inflammatory agent is disposed on the outer surface of the capsule.

15. The method of claim 12, wherein the anti-inflammatory agent is a member selected from the group consisting of a steroid, an NSAID and a combination thereof.

16. The method of claim 14, wherein the anti-inflammatory agent is a biocompatible polymer.

17. (canceled)

18. The method of claim 7, wherein the anti-inflammatory agent is co-administered locally.

19. (canceled)

20. The method of claim 7, wherein the anti-inflammatory agent is a member selected from the group consisting of a steroid, an NSAID and a combination thereof.

21. (canceled)

22. The method of claim 12, wherein the anti-inflammatory agent is co-administered locally.

23-25. (canceled)

26. The method of claim 7, wherein the AUC is increased by at least 10%.

27-30. (canceled)
